(19) United States
(12) Patent Application Publication
(10) Pub. No.: US 2009/0012499 A1
(43) Pub. Date: Jan. 8, 2009

(54) NOVEL METHOD FOR TREATING CHRONIC SEVERE HEART FAILURE BY USING INSULIN-LIKE GROWTH FACTOR-1 (IGF-1)

(75) Inventors: Kunio Miyatake, Osaka (JP); Kazuo Komamura, Nara (JP); Sumio Kiyoto, Kanagawa (JP)

Correspondence Address: OBLON, SPIVAK, MCCLELLAND MAIER & NEUSTADT, P.C., 1940 DUKE STREET, ALEXANDRIA, VA, 22314, US

(73) Assignees: Japan as Rep. by Pres. Of Nat. Cardiovascular Ctr., Osaka (JP); Astellas Pharma Inc., Tokyo (JP)

(21) Appl. No.: 11/815,684

(22) PCT Filed: Feb. 6, 2006
(86) PCT No.: PCT/JP2006/302405

§ 371 Date: Feb. 14, 2008

Related U.S. Application Data
(60) Provisional application No. 60/650,522, filed on Feb. 8, 2005.

(51) Int. Cl. A61M 31/00; A61B 5/00

(52) U.S. Cl. 604/522; 600/365

(57) ABSTRACT

The present invention relates to novel method for treating chronic severe heart failure by using insulin-like growth factor-1 (IGF-1). More specifically, the present invention relates to method for improving cardiovascular function and symptoms in a patient with chronic severe heart failure by administering IGF-1 to the patient for a certain period, wherein no options for treating the disorder the patient remain other than heart transplantation.

### TECHNICAL FIELD

The present invention relates to novel method for treating chronic severe heart failure by using insulin-like growth factor-1 (IGF-1). More specifically, the present invention relates to method for improving cardiovascular function and symptoms in a patient with chronic severe heart failure by administering IGF-1 to the patient for a certain period of time, wherein no options for treating the disorder of the patient remain other than heart transplantation.

### BACKGROUND ART

Many medical agents for treating heart failure have been put to practical use and contributed to improve survival and QOL in patients. For example, the beneficial effects of growth hormone (GH) on heart failure have been implicated by previous studies (Colao A et al, Clin Endocrinol (Oxf). 2001; 54:137-54, and Ren J et al, J Mol Cell Cardiol. 1999; 31:2049-61). Furthermore, currently, the use of angiotensin-converting-enzyme inhibitors (ACE-I) and beta-blockers has been very effective in treating the majority of patients with heart failure.

However, in randomized, double-blind trials, GH failed to improve cardiovascular function and symptom of heart failure in patients with idiopathic dilated cardiomyopathy (DCM) (Osterziel K J et al, Lancet. 1998; 351:1233-7, and Isgaard J et al, Eur Heart J. 1998; 19:1704-11). Furthermore, detailed analysis of that study revealed that sufficient doses of GH, proportional to body weight, are required for clinical effectiveness (Osterziel K J et al, J Clin Endocrinol Metab. 2000; 85:1533-9). Also, intractable, severe heart failure does not respond to ACE-I or beta-blockers.

Muscle wasting in heart failure deteriorates patient's QOL, and further, it will be a risk for mortality in chronic heart failure (Anker S D et al. Lancet. 1997; 349:1050-3). Skeletal muscle volume and strength were reduced along with the reduction in serum level of IGF-1 (Niebauer J et al. J Am Coll Cardiol. 1998; 32:393-7) and reduction in IGF-1 mRNA expression in skeletal muscle (Hambrecht R et al. J Am Coll Cardiol. 2002; 39:1175-81.) in chronic heart failure. Furthermore, low level of IGF-1 relates to rise of risk for occurrence of heart failure, myocardial infarction, or diabetes (Ann Intern Med 2003; 139:642-8, J Cardiol 2004; 94:384-6, Diabetes 2001; 50:637-42).

However, as severe GH resistance (Anker S D et al. J Am Coll Cardiol. 2001; 38:443-52, and Cicoira M et al. J Card Fail. 2003; 9:219-26.) and insulin resistance (Swan J W et al. J Am Coll Cardiol. 1997; 30:527-32, and Witteles R M et al. J Am Coll Cardiol. 2004; 44:78-81) are associated with chronic heart failure, exogenous GH or its insulin-like active mediator IGF-1 are supposed to be unlikely to affect cardiac and exercise performance in severe chronic heart failure. Furthermore, recently, it has been reported that insulin resistance is closely related to occurrence and development of heart failure (JAMA 2005; 294: 334-41) and that heart failure condition raises up insulin resistance (Am J Hypertens 2005; 18:731-7), however, it also has been reported that a representative drug for improving insulin resistance, thiazolidine derivative, leads to occurrence of heart failure (Lancet 2005; 366:1279-89).

That is, there are still several types of chronic severe heart failure of which the symptom can not be improved by using current medical agents as described above. At this time, for improving survival in patients with such type of severe heart failure, there is no choice other than heart transplantation. The number of patients on waiting list for heart transplantation increases for years, and there are also patients with chronic severe heart failure who have not been registered for waiting list for heart transplantation. Patients with severe heart failure requiring heart transplantation have not good prognosis, and one-year survival for such patients are only about 50 percent. Because of a shortage of donor hearts, waiting periods are growing longer, new therapeutic method other than heart transplantation must be developed.

### SUMMARY OF THE INVENTION

In view of the present situation described above, the present inventors have established novel method for treating chronic severe heart failure in case that no treatment option remain other than heart transplantation, by using insulin-like growth factor-1 (IGF-1).

Mecasermin (Somazon®, Fujisawa Pharmaceutical Co., Ltd., Osaka, Japan) is a genetically recombinant insulin-like growth factor-1 (IGF-1), an orphan drug that was developed in Japan for growth-hormone-resistant GH-deficiency and Laron-type dwarfism. Its safety and effective dosage have been verified in the treatment of dwarfism. In addition to cellular proliferating effects, IGF-1 has been demonstrated to possess positive inotropic, vasodilatory and anti-apoptotic actions in both cultured cells and in animals (Ren J et al, J Mol Cell Cardiol. 1999; 31:2049-61, McMullen J R et al, J Biol Chem. 2004; 279:4782-4793, von Lewinski D, et al, Circ Res. 2003; 92:169-76, Welch S et al, Circ Res. 2002; 90:641-8, Anwar A et al, Circulation. 2002; 105:1220-5, Yamashita K et al, Circ Res. 2001; 88:609-14, Fujio Y et al, Circulation. 2000; 101:660-7, Wang L et al, Circ Res. 1998; 83:516-22, Cittadini A et al, Circ Res. 1998; 83:50-9, Li Q et al, J Clin Invest. 1997; 100:1991-9). Acute injection of IGF-1 into healthy men and patients with heart failure resulted in significant inotropic effects without adverse effects (U.S. Pat. No. 5,434,134, Donath MY et al, J Clin Endocrinol Metab. 1996; 81:4089-94, and Donath M Y et al, J Clin Endocrinol Metab. 1998; 83:3177-83). However, to date, the therapeutic effect of chronic administration of IGF-1 for heart failure has not been reported.

The present inventors have established the present invention based on the novel knowledge that chronic administration of IGF-1 for chronic severe heart failure has significant therapeutic effect on the disease and therapeutic effects persists after the cessation of IGF-1 treatment.

In one aspect, the present invention provides a method for improving cardiovascular function and symptoms in a patient with chronic severe heart failure, wherein the method comprises administering therapeutically effective amount of IGF-1 to the patient for a certain period of time, and the improvement of cardiovascular function and symptoms in the patient persists after the cessation of IGF-1 treatment. In one embodiment, the method of the present invention further comprises, in the certain period of time for administration, a) monitoring the level of blood sugar in the patient, and b) administering sugars to the patient.

In another aspect, the present invention provides a pharmaceutical composition for improving cardiovascular function and symptoms in a patient with chronic severe heart failure, comprising therapeutically effective amount of IGF-1.

In another aspect, the present invention provides a use of IGF-1 for manufacturing a medicament for improving cardiovascular function and symptoms in a patient with chronic severe heart failure.

The method of the present invention can improve severity of symptoms, QOL, cardiovascular-function, vascular function, exercise tolerance, etc. The present invention also enables removal of left ventricular assist device (LVAD) from patient and/or withdrawal from the waiting list for heart transplant.

### BRIEF DESCRIPTION OF THE DRAWINGS

FIG. 1 shows individual responses of key parameters to IGF-1.

FIG. 2 shows representative histological images, images of staining with Masson-trichrome, IGF-L antibody, IGF-L receptor antibody, phospholylated Akt antibody, TNF-A antibody and TUNEL at baseline, at the end of treatment, and at the follow-up from a single patient.

FIG. 3 shows representative cardiac magnetic resonance images and dot plots of CD34⁺ cells.

### DETAILED DESCRIPTION OF THE PRESENT INVENTION

The present invention is now described in detail.

The heart failure that can be treated by the method of the present invention includes, but not to be limited, several types of chronic severe heart failure such as dilated cardiomyopathy (DCM), dilated form of hypertrophic cardiomyopathy, ischemic cardiomyopathy, and ischemic heart disease. Improvement of cardiovascular function and symptoms in the patient can be confirmed by determining a variety of index associated with cardiovascular function and symptoms known in the art using conventional techniques.

In one embodiment of the present invention, the number of circulating CD34⁺ endothelial progenitor cells (EPC) in the patient is increased. CD34⁺ EPC is a type of stem cells which has ability of differentiating into myocardium cells or vascular cells, which strengthen cardiovascular function in the patient.

In one embodiment, the chronic severe heart failure that can be treated by the method of the invention is accompanied by insulin resistance condition. Most types of chronic severe heart failure are associated with increase of insulin resistance, and the method of the invention can improve insulin resistance with cardiovascular function by administering IGF-1 for a certain period of time.

In one embodiment, the patient who is particularly expected to be treated by the method of the present invention is a patient with chronic severe heart failure who requires heart transplantation. Chronic severe heart failure, which heart transplantation is required to treat, include dilated cardiomyopathy, dilated form of hypertrophic cardiomyopathy, and other heart failure decided to be treated by heart transplantation. Whether a patient with heart failure requires heart transplantation or not is decided by physicians based on defined criteria for heart transplantation. Representatively, the patient requiring heart transplantation who is expected to undergo the treatment by the present invention includes patients classified in New York Heart Association (NYHA) functional class III through IV. In one embodiment, chronic severe heart failure that can be more effectively treated by the method of the present invention is dilated cardiomyopathy.

The IGF-1 useful for the present invention includes, but is not limited to, human IGF-1 and analogue thereof. Preferably, the IGF-1 useful for the present invention is recombinant IGF-1 Mecasermin (Somazon®, Fujisawa Pharmaceutical Co., Ltd., Osaka, Japan).

The administration period of IGF-1 and the number of times of administration of IGF-1 may increase or decrease according to instruction of physician considering the symptom, age and body weight of the patient, administration route etc. The certain period of time for administration in the present invention is any period after which the improvement of cardiovascular function and symptoms in the patient persists. In one embodiment, the period of time is at least about 1 month, preferably at least 2 months, and more preferably at least 3 months. For example, the period is about 1 to 6 months, preferably about 2 to 4 months, and more preferably about 3 months. The number of times of administration of IGF-1 is, for example, twice a day to once a week, and preferably about once a day in the period.

The therapeutically effective amount of IGF-1 to be used in the method of the invention means the amount of IGF-1 needed for improving cardiovascular function and symptom of heart failure in the patient, and is preferably 0.01 mg/kg to 0.5 mg/kg and more preferably 0.05 mg/kg to 0.3 mg/kg. However, according to instruction of physician considering the symptom, age and body mass of the patient, administration route etc., the amount of IGF-1 to be administered to a patient may increase or decrease. In preferable embodiment, in order to prevent hypoglycemia which may occurred by hypoglycemic effect of IGF-1, the amount of IGF-1 administered to the patient is titrated over the period of time for administration (e.g., daily, weekly, or monthly).

The administration route of IGF-1 to be used in the method of the invention includes, but is not limited to, subcutaneous, intramuscular, intravenous, nasal, oral, dermal or a combination thereof. Preferably, the administration route is subcutaneous.

A composition comprising IGF-1 to be administered to a patient has any form of pharmaceutical preparation well known in the art, for example, in liquid, solid or semisolid form, which contains IGF-1, as an active ingredient, in admixture with an organic or inorganic carrier or excipient suitable for subcutaneous, intramuscular, intravenous, nasal, oral, dermal, parenteral or any other pharmaceutical application. IGF-1 can be formulated, for example, with the conventional non-toxic, pharmaceutically acceptable carriers for liquids, solution, emulsion, suspension, injections, liposome inclusions, powders, capsules, pellets, tablets, and any other form suitable for use. The carriers which can be used are water, saline, wax, sugar and other carriers suitable for use in manufacturing preparations in liquid, solid or semisolid form, with or without additional additives such as auxiliary, stabilizing, and isotonizing agent.

In the method of the present invention, IGF-1 may also be administered to a patient in combination with other agents simultaneously, separately, or sequentially through the same or different administration route. For example, in the method of the present invention, IGF-1 may administered to a patient in combination with sugar such as glucose, sucrose, maltose, lactose, and oligosaccharide to compensate drop in glucose level by hypoglycemic effect of IGF-1, or in combination with other known drug for improving the condition of a patient with heart failure such as growth hormone, vasodilators, ACE-inhibitor, β-blocker, calcium antagonists, antiarrhythmic drugs, cardiotonic drug, and diuretic. In one embodiment, the method of the present invention further comprises, in the certain period of treatment, a) monitoring the level of blood sugar in the patient, and b) administering sugars to the patient.

The following example illustrates treating dilated cardiomyopathy using IGF-1 and as such is not to be considered as limiting the present invention in the appended claims.

### EXAMPLE

### Method

### Study Protocol

The study was conducted according to the declaration of Helsinki and was approved by the institutional review boards. After approval by the institutional ethics committee, consecutive eight patients were enrolled into the study. Eligibility requirements were as follows: 1) diagnosis of DCM based on the definition of the World Health Organization/International Society and Federation of Cardiology Task Force (Richardson P et al. Circulation. 1996; 93:841-2), confirmed by cardiac catheterization and endomyocardial biopsy; 2) New York Heart Association (NYHA) functional class III or IV and left ventricular (LV) ejection fraction (EF) less than 30% despite of optimal treatment including left ventricular assist device (LVAD); and 3) registration in the Japan Organ Transplant Network. Exclusion criteria were as follows: 1) acute or unstable stage of heart failure; 2) heart failure due to primary ischemic or valvular heart diseases, or myocarditis; 3) malignancy; 4) tendency to hypoglycemia due to various disorders including diabetes mellitus; 5) renal and/or hepatic dysfunctions; 6) disorders in endocrine system including GH and IGF-1; and 7) infectious or inflammatory diseases. Baseline characteristics of the patients are reported in Table 1. As shown in Table 1, all the patients received optimal and maximal conventional medications for refractory heart failure. Of note, effective dose of carvedilol for Japanese patients with heart failure was 5 to 20 mg in MUCHA study (Hori M et al. Am Heart J. 2004; 147:324-30), which was less than half of that in MOCHA study (Bristow M R et al. Circulation. 1996; 94:2807-16) in United States. None of them had abnormal levels of serum GH and IGF-1. After written informed consent was obtained, recombinant IGF-1 was given to eight patients for three months in addition to the other previous medications. Treatments other than IGF-1 were not changed during the three-month injection period. Subcutaneous IGF-1, injected once a day at 0900 h after breakfast, was titrated weekly from 0.05 mg/kg up to 0.3 mg/kg with close monitoring of blood glucose in order to avoid hypoglycemia. The present inventors set the dose range from 0.05 to 0.3 mg/kg, according to the approved dose for insulin receptor abnormality or Laron dwarfism in Japan. The final dose was determined not to induce hypoglycemia. Final doses ranged from 0.15 to 0.30 mg/kg (median 0.25 mg/kg).

TABLE 1
Patient characteristic

| | Patient | | | | | | | |
|---|---|---|---|---|---|---|---|---|
| | #1 | #2 | #3 | #4 | #5 | #6 | #7 | #8 |
| Age (year) | 37 | 25 | 27 | 35 | 54 | 34 | 36 | 35 |
| Sex | Male | Male | Male | Male | Male | Female | Male | Male |
| NYHA | III | III | IV | III | IV | IV | III | III |
| functional | | | | | | | | |
| class | | | | | | | | |
| Ejection | 18 | 21 | 8 | 17 | 28 | 19 | 6 | 24 |
| Fraction (%) | | | | | | | | |
| Duration of | 7.8 | 12.9 | 1.0 | 5.6 | 3.9 | 7.7 | 4.9 | 1.5 |
| CHF (year) | | | | | | | | |
| Family | (+) | (−) | (+) | (+) | (−) | (+) | (+) | (+) |
| history of | | | | | | | | |
| DCM | | | | | | | | |
| Waiting list | Status 2 | Status 2 | Status 1 | In preparation | Status 1 | Status 1 | Status 2 | Status 2 |
| for HTx | | | | | | | | |
| IGF-1 max | 0.20 | 0.23 | 0.30 | 0.15 | 0.24 | 0.30 | 0.27 | 0.26 |
| dose | | | | | | | | |
| (mg/kg/day) | | | | | | | | |

Treatment (dose (mg), (duration before treatment (months))

| | | | | | | | | |
|---|---|---|---|---|---|---|---|---|
| BB | CRV 15 (25) | MTP 40 (24) | CRV 30 (11) | CRV 15 (26) | CRV 2.5 (23) | MTP 10 (27) | CRV 10 (30) | BSP 5 (15) |
| ACEI | ENL 2.5 (34) | ENL 5(42) | ENL 2.5 (12) | ENL 5 (26) | | | | ENL 2.5 (15) |
| ARB | | | | | CDS 2 (23) | LOS 25 (24) | VLS 20 (26) | |
| ALD | SPR 25 (34) | SPR 75 (42) | SPR 25 (12) | SPR 50 (26) | SPR 25 (23) | SPR 25 (27) | SPR 25 (30) | SPR 25 (15) |
| DUR | FRS 40 (34) | FRS 60 (42) | FRS 40 (12) | FRS 40 (26) | FRS 80 (23) | FRS 60 (27) | FRS 80 (30) | FRS 80 (15) |
| | | | | | TCM 8 (23) | AZS 60 (27) | | AZS 60 (15) |
| NTR | | | | ISDN 20 (26) | | | | |
| ART | AMD 150 (15) | VRP 160 | AMD 100 (11) | AMD 100 (7) | AMD 100 (23) | AMD 100 (27) | AMD 150 (30) | AMD 100 (12) |
| | | (25) | | | | MEX 100 (27) | | |
| DIG | DGX 0.125 | | DGX 0.125 | DGX 0.25 (26) | DGX 0.125 (23) | DGX 0.125 | DGX 0.125 | DGX 0.125 (15) |
| | (34) | | (12) | | | (27) | (30) | |
| Oral INT | PMB 2.5 (10) | | | PMB 2.5 (3) | | PMB 3.75 (12) | PMB 2.5 (9) | PMB 2.5 (10) |
| IV INT (γ) | | | | | MLR 0.2 (2) | DOB 3 (2) | | |
| | | | | | | MLR 0.5 (2) | | |
| LVAD | | | TOYOBO (3) | | | | | |

ACEI = angiotensin converting enzyme inhibitor;
ARB = angiotensin receptor blocker;
ALD = aldosterone inhibitor;
AMD = amiodarone;
ART = antiarrhythmic drugs;
AZS = azosemide;
BB = beta blocker;
BSP = bisoprolol;
CDS = candesartan;
CHF = chronic heart failure;
CRV = carvedilol;
DCM = dilated cardiomyopathy;
DGX = digoxin;
DIG = digitalis;
DOB = dobutamine;
DUR = diuretics;
ENL = enalapril;
FRS = furosemide;
HTx = heart transplantation;
INT = inotropics;
ISDN = isosorbite dinitrate;
IV = intravenous;
LOS = losartan;
MEX = mexiletine;
MLR = milrinone;
MTP = metoprolol;
NTR = nitrate;
PMB = pimobendan;
SPR = spironolactone;
TCM = trichlormethiazide;
TOYOBO; extracoporeal assist device VCT-50 made by TOYOBO;
VLS = valsartan;
VRP = verapamil

### Cardiac Function, Exercise Capacity and Blood Neurohumoral Factors

Before the beginning of the IGF-1 treatment, at its end, and three months after its discontinuation, the NYHA functional class and daily life activity assessed by the specific activity scale questionnaire were determined in all patients (Sasayama S et al. Evaluation of functional capacity of patients with congestive heart failure. In: Yasuda H, Kawaguchi H eds. New Aspects in Treatment of Failing Heart. Springer-Verlag; 1992:113-7). At the same time points, cardiac catheterization, echocardiography, magnetic resonance imaging (MRI), cardiopulmonary exercise test, high-resolution ultrasound flow-mediated dilatation (FMD) of the brachial artery, and coronary flow velocity reserve (CFVR) under sinus rhythm assessed by transthoracic Doppler echocardiography were conducted. For a patient with LVAD, enhanced electron beam CT was performed instead of MRI (Naito H et al. Invest Radiol. 1992; 27:436-42). Also at those time points, the estimation of lean body mass by dual-energy X-ray absorptiometry (Lunar DPX-L) was available in five patients. Likewise, blood levels before breakfast of the following substances were measured: standard laboratory chemistry, complete blood counts, IGF-1 (Daiichi Radioisotope), free IGF-1 (Takada M et al. J Immunoassay. 1994; 15:263-76.) (Mitsubishi Kagaku Iatron Inc.), GH (Daiichi Radioisotope), IGF binding protein-3 (IGFBP-3) (Diagnostic Systems Laboratories), atrial natriuretic peptide (ANP) (Shionogi), brain natriuretic peptide (BNP) (Shionogi), epinephrine, norepinephrine, renin activity, aldosterone, angiotensin II, cyclic GMP (Cayman Chemical), nitrite/nitrate (HPLC autoanalyzer, Eicom), interleukin-6 (R&D Systems), and tumor necrosis factor-α (TNF-α) (R&D Systems).

Because IGF-1 stimulates proliferation of putative endothelial progenitor cells (EPC) such as CD34⁺ cells (Frostad S et al. Stem Cells. 1998; 16:334-42), circulating CD34⁺ and CD133⁺ cells were counted with standard flow cytometry method (Taguchi A et al. Circulation. 2004; 109:2972-5).

### Histology of Myocardial Biopsies

Myocardial biopsies were obtained from the right ventricular side of the septum at the abovementioned three-time points. Five-micrometer-thick paraffin embedded sections were stained and examined with microscopic digital camera in random order by observers who were blinded to the sample source and date of acquisition. Myocyte diameter and percent fibrosis area were determined by a digital image analyzer according to the method described previously (Komamura K et al. Hypertension. 2004; 44:365-71). Immunohistochemical stainings were performed using an anti-human IGF-I monoclonal antibody (Upstate Biotechnology), anti-human IGF-1 receptor (IGF-1R) monoclonal antibody (R&D Systems), anti-human phosphorylated Akt monoclonal antibody (Cell Signaling) and anti-human TNF-A monoclonal antibody (Santa Cruz). The myocardial contents of IGF-1, IGF-1R, phosphorylated Akt kinase and TNF-α were determined by quantitative analysis of stained area using Win-ROOF (Mitani) according to the method by Torre-Amione et al (Kucuker S A et al. J Heart Lung Transplant. 2004; 23:28-35) based on Matsuo's method (Matsuo T et al Histochem J. 1995; 27:989-96). Terminal dUTP nick-end labeling (TUNEL) staining was performed with ApopTag Kit (Chemicon International) with counter staining by methyl green. Cardiomyocytes in 60 random high-power fields (×400) per patients were counted.

### Myocardial Gene Expressions

Total RNA was prepared from biopsy samples using an RNeasy Fibrous Tissue Kit (QIAGEN). From these samples, mRNA was then amplified by T7 RNA polymerase. Complementary DNA was produced by real-time quantitative reverse-transcription polymerase chain reaction (RT-PCR) using a QuantiTect RT-PCR Kit (QIAGEN) and by a Prism 7700 Sequence Detector (Applied Biosystems) (Komamura K et al. Cardiovasc Drugs Ther. 2003; 17: 303-10). To correct for the efficiency of cDNA synthesis, measured mRNA amounts were divided by the amount of glyceraldehyde-3-phosphate-dehydrogenase (GAPDH) mRNA as an internal standard. The inventors used the following sense and antisense primers (Takara Bio Inc., Otsu): 5′-GCTGAGCTGGTGGATGCTCTT-3′ and 5′-TCATCCACGATGCCTGTCTG-3′ for IGF-1 (116 bp); 5′-ACCAGGGCTTGTCCAACGAG-3′ and 5′-AGCACATGCGCATCAGTTCA-3′ for IGF-1R (108 bp); 5′-CACAGTGAAGTGCTGGCAAC-3′ and 5′-TTCCAGATGTCAGGGATCAAAG-3′ for TNF-α (89 bp); 5′-CCTGCACCACCAACTGCTTAG-3′ and 5′-GCCATCACGCCACAGTTTC-3′ for GAPDH (146 bp). Sequences of amplified fragments were confirmed by DNA sequencing.

### Statistics

Data are presented as mean±SD unless indicated otherwise. Changes in variables between the baseline and the end of treatment, and the baseline and the three-month follow-up were evaluated by either paired t test or Wilcoxon signed-rank test, as appropriate. The correlation of the changes in variables was assessed by Pearson or Spearman correlation coefficient, as appropriate. A value of p<0.05 was considered significant.

### Results

### Symptoms and Cardiac Function

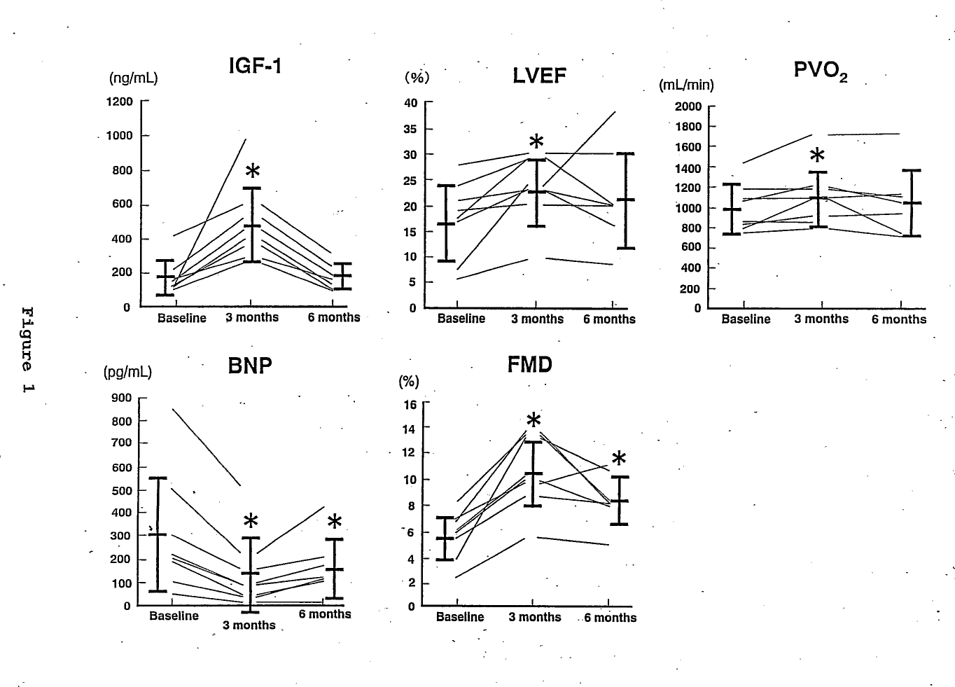

None of the patients died or underwent heart transplantation during the treatment and the follow-up period. One patient, whose cardiac performance responded to the treatment, had an unexpected relapse of heart failure after completion of treatment. His follow-up study was not conducted because of unstable conditions. Symptoms and physiological parameters of the patients at baseline, at the end of treatment, and at the three-month follow-up are described in Table 2 and FIG. 1. NYHA functional class and daily life activity were significantly improved at the end of treatment, and remained improved at the follow-up. Blood pressure, heart rate, number of ventricular arrhythmias, and cardiothoracic ratio did not change significantly during the entire study. Bodyweight and lean body mass were increased after treatment and then returned to baseline levels at the follow-up.

Five out of eight patients (Patients 1-4 and 8) experienced symptomatic hypoglycemia just before lunch during the titerating period of IGF-1. The final dose was determined not to induce hypoglycemia. Neither consciousness loss nor faintness was reported. Patients 2 and 6 had transient increases in hepatic transaminases, which were normalized within two weeks with no interruption of IGF-1 injection. In all the patients, blood levels of IRI, CPR, ACTH, FSH, LH, T₄, T₃, CEA, AFP, CA19-9, BCA225, and PA were not changed during the IGF-1 treatment.

Catheter-derived cardiac index was increased, and LV end-diastolic pressure and systemic vascular resistance index were decreased significantly at the end of treatment. At the end of treatment, echocardiographic fractional shortening and posterior wall thickness increased significantly, without further deterioration of diastolic function, which was determined by E/A and deceleration time of E wave (Table 2). Likewise, LVEF and LV mass index (LVMI), determined by MRI, were significantly increased. The percent changes in LVEF correlated with the percent changes in LVMI (p=0.043, r=0.72). Variables of exercise capacity such as peak oxygen consumption, anaerobic threshold, exercise duration, and peak workload were significantly increased compared to baseline. FMD and CFVR were also increased significantly. The percent changes in FMD correlated with the percent changes in peak oxygen consumption (p=0.021, r=0.79). At three-month follow-up, those physiological parameters, except for functional class, daily life activity, wall thickness and FMD, had returned to baseline levels (Table 2 and FIG. 1).

TABLE 2
Symptoms and Physiological Parameters

| Variables | | Baseline | n | End of treatment | n | p vs. base | 3 Months F/U | n | p vs. base |
|---|---|---|---|---|---|---|---|---|---|
| NYHA class | | 3.4 ± 0.5 | 8 | 2.3 ± 0.5 | 8 | 0.012 | 2.1 ± 0.4 | 7 | 0.018 |
| Specific Activity | Mets | 3.0 ± 0.9 | 8 | 5.3 ± 1.2 | 8 | 0.012 | 5.4 ± 0.8 | 7 | 0.018 |
| Scale | | | | | | | | | |
| SBP | mmHg | 98.5 ± 4.4 | 8 | 96.9 ± 3.7 | 8 | 0.089 | 98.6 ± 6.9 | 7 | 0.089 |
| DBP | mmHg | 57.5 ± 3.8 | 8 | 55.4 ± 3.9 | 8 | 0.14 | 56.4 ± 4.8 | 7 | 0.46 |
| Heart Rate | bpm | 67.1 ± 9.1 | 8 | 74.0 ± 8.8 | 8 | 0.15 | 69.4 ± 6.9 | 7 | 0.59 |
| VPCs per day | beats | 2634 ± 2674 | 8 | 2575 ± 2884 | 8 | 0.83 | 1532 ± 1918 | 7 | 0.063 |
| CTR | % | 53.2 ± 3.9 | 8 | 51.4 ± 5.1 | 8 | 0.45 | 52.6 ± 3.7 | 7 | 0.76 |
| Body Weight | kg | 55.4 ± 9.7 | 8 | 59.7 ± 9.8 | 8 | <0.0001 | 56.8 ± 10.6 | 7 | 0.090 |
| Lean Body Mass | kg | 39.7 ± 8.1 | 5 | 43.2 ± 8.2 | 5 | 0.0005 | 39.1 ± 7.5 | 4 | 0.25 |
| Fat Weight | kg | 8.4 ± 2.9 | 5 | 8.5 ± 3.1 | 5 | 0.90 | 9.4 ± 2.4 | 4 | 0.39 |
| Average Grip Force | kg | 26.2 ± 6.8 | 5 | 28.6 ± 7.5 | 5 | 0.0053 | 27.1 ± 6.7 | 4 | 0.31 |
| Catheterization | | | | | | | | | |
| mean AP | mmHg | 77.8 ± 2.6 | 8 | 76.8 ± 2.4 | 8 | 0.41 | 77.0 ± 2.7 | 7 | 0.21 |
| Cardiac Index | L/min/m² | 2.8 ± 0.8 | 8 | 3.3 ± 0.8 | 8 | 0.013 | 2.9 ± 0.5 | 7 | 0.94 |
| LVEDP | mmHg | 14.3 ± 8.9 | 8 | 11.0 ± 8.6 | 8 | 0.043 | 10.6 ± 8.3 | 7 | 0.094 |
| SVRI | dyne · sec · | 2041.0 ± 521.9 | 8 | 1637.4 ± 380.9 | 8 | 0.004 | 1868.1 ± 259.2 | 7 | 0.24 |
| | cm⁻⁵ · m² | | | | | | | | |
| Echocardiography | | | | | | | | | |
| EDD | mm | 73.3 ± 11.4 | 8 | 70.4 ± 12.2 | 8 | 0.17 | 73.3 ± 9.8 | 7 | 0.48 |
| FS | % | 9.0 ± 5.6 | 8 | 13.9 ± 5.8 | 8 | 0.015 | 13.3 ± 4.4 | 7 | 0.080 |
| PW thickness | mm | 6.0 ± 0.8 | 8 | 6.9 ± 1.0 | 8 | 0.0002 | 6.6 ± 1.3 | 7 | 0.030 |
| E/A | | 1.38 ± 0.58 | 7 | 1.36 ± 0.53 | 7 | 0.90 | 1.11 ± 0.52 | 6 | 0.50 |
| Deceleration time | msec | 151.3 ± 35.2 | 8 | 152.8 ± 46.5 | 8 | 0.94 | 166.1 ± 31.6 | 7 | 0.51 |
| MR tomography | | | | | | | | | |
| EDVI | mL/m² | 199.4 ± 87.7 | 8 | 194.0 ± 92.5 | 8 | 0.25 | 202.1 ± 111.4 | 7 | 0.80 |
| ESVI | mL/m² | 167.3 ± 87.4 | 8 | 153.3 ± 90.7 | 8 | 0.012 | 163.0 ± 108.7 | 7 | 0.35 |
| LVEF | % | 17.6 ± 7.5 | 8 | 23.4 ± 6.7 | 8 | 0.016 | 21.9 ± 9.5 | 7 | 0.26 |
| LV mass index | g/m² | 91.0 ± 22.4 | 8 | 98.5 ± 26.5 | 8 | 0.003 | 90.6 ± 14.6 | 7 | 0.14 |
| Exercise Capacity | | | | | | | | | |
| peakVO₂ | mL/min | 997.1 ± 228.2 | 8 | 1106.1 ± 284.4 | 8 | 0.033 | 1065.3 ± 334.3 | 7 | 0.34 |
| Anaerobic threshold | mL/min | 476.7 ± 103.0 | 7 | 558.4 ± 115.4 | 7 | 0.013 | 522.0 ± 121.0 | 7 | 0.11 |
| Exercise Duration | min | 7.7 ± 0.8 | 8 | 8.6 ± 1.1 | 8 | 0.010 | 8.5 ± 1.4 | 7 | 0.13 |
| Workload | W | 90.1 ± 22.2 | 8 | 99.6 ± 23.8 | 8 | 0.006 | 97.0 ± 31.4 | 7 | 0.28 |
| Vascular Doppler | | | | | | | | | |
| FMD | % | 5.63 ± 1.88 | 8 | 10.5 ± 2.87 | 8 | 0.0006 | 8.52 ± 2.08 | 7 | 0.013 |
| CFVR | | 1.76 ± 0.64 | 7 | 2.17 ± 0.55 | 7 | 0.024 | 1.75 ± 0.60 | 6 | 0.93 |

AP = aortic pressure;
CTR = cardiothoracic ratio;
CFVR = Coronary Flow Velocity Reserve;
DBP = diastolic blood pressure;
EDD = end-diastolic diameter;
EDVI = end-diastolic volume index;
ESVI= end-systolic volume index;
FMD = flow mediated diameter;
FS = fractional shortening;
LVEDP = left ventricular end-diastolic pressure;
LVEF = left ventricular ejection fraction;
MR = megnetic resonance;
PW = posterior wall;
SBP = systolic blood pressure;
SVRI = systemic vascular resistance index;
VO₂ = oxygen consumption;
VPC = ventricular premature beat.

### Blood Neurohumoral Factors

At the end of treatment, serum IGF-1 and free IGF-1 was increased significantly from their baseline levels, to which they had returned at the follow-up (Table 3). The percent changes in free IGF-1 level correlated with the percent changes in LVMI (p=0.028, r=0.83) and LVEF (p=0.010, r=0.976) during the three-month treatment. GH and IGFBP-3 levels were decreased significantly from their baseline levels, to which they had returned at the follow-up. At the same time points, levels of norepinephrine, ANP, BNP, IL-6, and TNF-A were decreased significantly. Renin, aldosterone and angiotensin II levels did not change. Cyclic GMP and nitrite/nitrate levels were increased significantly. The percent changes in serum cyclic GMP (p=0.044, r=0.76), nitrite/nitrate (p=0.032, r=0.81), IL-6 (p=0.044, r=−0.76) and TNF-α (p=0.017, r=−0.91.) levels correlated with the percent changes in FMD. An index of insulin resistance, HOMA-IR, improved at the end of treatment, which may relate to endothelial function. Putative endothelial progenitor cells were increased, which may relate to the improvement in FMD. At the follow-up, those neurohumoral parameters, except for BNP, cyclic GMP and nitrite/nitrate had returned to their baseline levels.

TABLE 3
Blood Neurohumoral Factors

| Variables | | Baseline | n | End of treatment | n | p vs. base | 3 Months F/U | n | p vs. base |
|---|---|---|---|---|---|---|---|---|---|
| Growth Factors | | | | | | | | | |
| Serum IGF-1 | ng/mL | 186 ± 105 | 8 | 484 ± 227 | 8 | 0.012 | 187 ± 81 | 7 | 0.92 |
| Serum free IGF-1 | ng/mL | 3.3 ± 1.7 | 8 | 10.3 ± 5.8 | 8 | 0.012 | 3.6 ± 3.1 | 7 | 0.61 |
| Serum IGFBP-3 | μg/mL | 2.35 ± 0.65 | 8 | 1.90 ± 0.61 | 8 | 0.017 | 2.50 ± 0.45 | 7 | 0.15 |
| Serum GH | ng/mL | 1.43 ± 1.63 | 8 | 0.48 ± 0.89 | 8 | 0.012 | 1.21 ± 0.45 | 7 | 0.25 |
| Hormones and Cytokines | | | | | | | | | |
| Plasma Epinephrine | pg/mL | 17 ± 14 | 8 | 14 ± 6 | 8 | 0.44 | 20 ± 18 | 7 | 0.93 |
| Plasma Norepinephrine | pg/mL | 804 ± 664 | 8 | 474 ± 250 | 8 | 0.036 | 577 ± 390 | 7 | 0.74 |
| Plasma ANP | pg/mL | 97 ± 64 | 8 | 55 ± 44 | 8 | 0.012 | 103 ± 63 | 7 | 0.15 |
| Plasma BNP | pg/mL | 304 ± 264 | 8 | 150 ± 168 | 8 | 0.012 | 166 ± 131 | 7 | 0.028 |
| Plasma Renin Activity | ng/mL/hr | 13.5 ± 4.3 | 8 | 14.2 ± 8.8 | 8 | 0.78 | 17.1 ± 8.0 | 7 | 0.31 |
| Plasma Aldosterone | pg/mL | 38.5 ± 32.7 | 8 | 30.5 ± 30.4 | 8 | 0.16 | 46.8 ± 49.4 | 7 | 0.50 |
| Plasma Angiotensin II | pg/mL | 58.9 ± 38.8 | 8 | 65.3 ± 78.3 | 8 | 0.58 | 65.9 ± 46.2 | 7 | 0.87 |
| Plasma cGMP | pmol/mL | 6.46 ± 2.19 | 8 | 9.80 ± 2.90 | 8 | 0.012 | 7.99 ± 3.32 | 7 | 0.043 |
| Serum Nitrite + Nitrate | μmol/L | 37.2 ± 9.4 | 8 | 56.4 ± 20.1 | 8 | 0.012 | 51.0 ± 22.3 | 7 | 0.028 |
| Serum IL-6 | pg/mL | 11.0 ± 4.31 | 8 | 4.35 ± 1.59 | 8 | 0.012 | 4.57 ± 3.18 | 7 | 0.018 |
| Serum TNF-α | pg/mL | 7.63 ± 3.01 | 8 | 3.04 ± 1.18 | 8 | 0.012 | 3.74 ± 2.02 | 7 | 0.018 |
| Insulin Resistance | | | | | | | | | |
| serum IRI | μU/mL | 7.36 ± 3.71 | 8 | 4.53 ± 2.18 | 8 | 0.059 | 8.01 ± 4.39 | 7 | 0.52 |
| plasma FBG | mg/dL | 87.4 ± 6.8 | 8 | 85.0 ± 7.0 | 8 | 0.20 | 86.7 ± 7.4 | 7 | 0.46 |
| HOMA-IR | | 1.60 ± 0.82 | 8 | 0.96 ± 0.48 | 8 | 0.047 | 1.73 ± 0.99 | 7 | 0.54 |
| Endothelial Progenitor Cells | | | | | | | | | |
| CD34⁺ | cells/μL | 1.79 ± 1.05 | 8 | 3.49 ± 1.44 | 8 | 0.039 | 1.80 ± 0.94 | 4 | 0.080 |
| CD133⁺ | cells/μL | 1.15 ± 0.71 | 8 | 2.10 ± 1.10 | 8 | 0.047 | 1.18 ± 0.33 | 4 | 0.21 |

ANP = atrial natriuretic peptide;
BNP = brain natriuretic peptide;
FBG = fasting blood glucose
cGMP = cyclic guanosine 3′,5′-monophosphate;
GH = growth hormone;
HOMA-IR = homeostasis model assessment insulin resistance;
IGF = insuline-like growth factor;
IGFBP = insuline-like growth factor biding protein;
IL-6 = interleukin 6;
IRI = immunoreactive insulin;
TNF-α = tumor necrosis factor α

### Quantification of Myocardial Biopsies

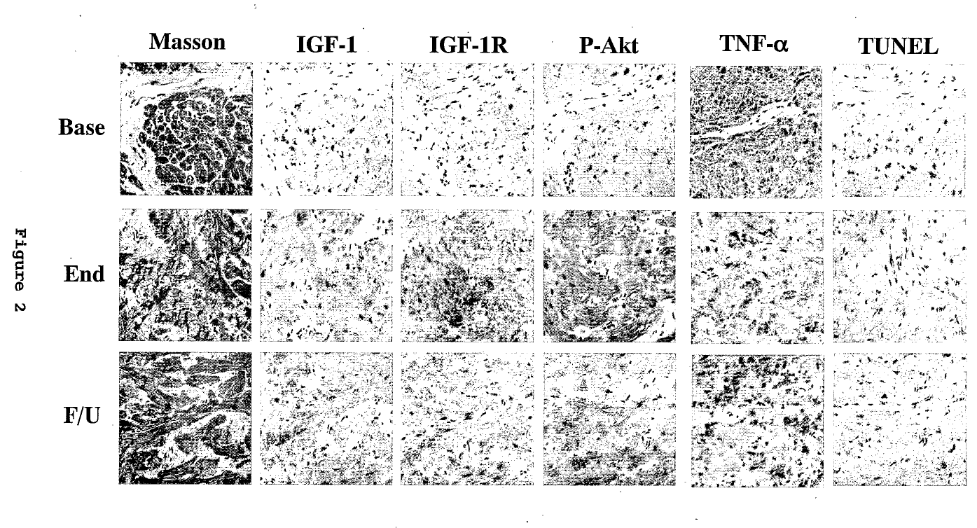

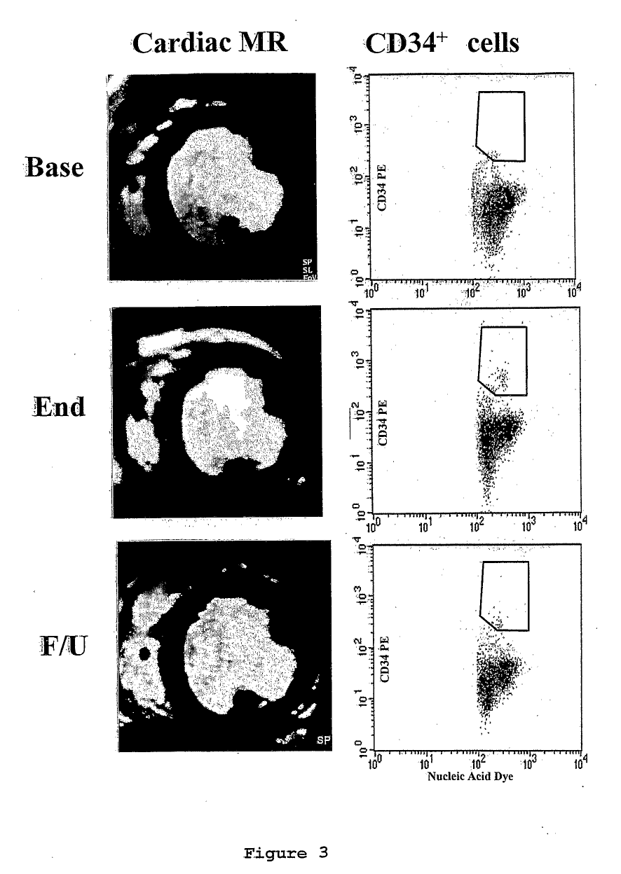

Myocyte diameter was increased significantly at the end of treatment (Table 4). Area of fibrosis did not change during the entire study. Percent changes in myocyte diameter correlated with the percent changes in LVMI (p=0.017, r=0.91) at the end of treatment. The changes in fibrosis area did not correlated with the changes in LVMI, suggesting myocardial not fibrous hypertrophy underlay LV hypertrophy. Staining area of IGF-1 and IGF-1R expanded larger at the end of treatment. At the same time, expansion of the staining area of phosphorylated Akt kinase and diminution of the staining area of TNF-A were observed. Concomitantly, gene-expression levels of IGF-1 and IGF-1R increased, and the expression level of TNF-α decreased (Table 4). The percentage of TUNEL positive cells tended to decrease at the end of treatment. FIG. 2 shows representative images of myocardial histology. FIG. 3 shows magnetic resonance imaging of ventricular structure and changes in counts of putative circulating EPC.

TABLE 4
Quantification of Myocardial Biopsies

| Variables | | Baseline | n | End of treatment | n | p vs. base | 3 Months F/U | n | p vs. base |
|---|---|---|---|---|---|---|---|---|---|
| Histology | | | | | | | | | |
| Myocyte diameter | μm | 27.8 ± 3.6 | 8 | 30.5 ± 4.5 | 8 | 0.012 | 28.8 ± 3.3 | 7 | 0.735 |
| Fibrotic area | % | 27.7 ± 6.2 | 8 | 28.1 ± 5.8 | 8 | 0.575 | 28.8 ± 6.2 | 7 | 0.128 |
| Immunohistichemistry | | | | | | | | | |
| IGF-1 positive area | % | 9.24 ± 1.34 | 8 | 13.5 ± 3.87 | 8 | 0.012 | 11.4 ± 1.89 | 7 | 0.063 |
| IGF-1R positive area | % | 4.69 ± 1.81 | 8 | 8.84 ± 6.11 | 8 | 0.036 | 8.02 ± 4.66 | 7 | 0.091 |
| P-Akt positive area | % | 1.34 ± 0.54 | 8 | 10.7 ± 4.96 | 8 | 0.012 | 1.65 ± 0.51 | 7 | 0.063 |
| TNF-α positive area | % | 40.3 ± 15.5 | 8 | 19.0 ± 10.5 | 8 | 0.012 | 29.8 ± 15.7 | 7 | 0.043 |
| TUNEL positive cells | % | 42.9 ± 11.7 | 8 | 39.0 ± 11.1 | 8 | 0.063 | 41.8 ± 12.4 | 7 | 0.999 |
| Real Time RT-PCR | | | | | | | | | |
| IGF-1/GAPDH | | 0.73 ± 0.15 | 8 | 1.53 ± 0.64 | 8 | 0.012 | 1.03 ± 0.57 | 7 | 0.063 |
| IGF-1R/GAPDH | | 0.68 ± 0.20 | 8 | 1.44 ± 0.84 | 8 | 0.012 | 0.92 ± 0.43 | 7 | 0.237 |
| TNF-α/GAPDH | | 1.67 ± 0.45 | 8 | 1.29 ± 0.59 | 8 | 0.017 | 1.38 ± 0.59 | 7 | 0.063 |

GAPDH = glyceraldehyde 3 phosphate dehydrogenase;
IGF-1 = insuline-like growth factor-1;
IGF-1R = insuline-like growth factor-1receptor;
P-Akt = phosphorylated Akt;
TNF-α = tumor necrosis factor-α;
TUNEL = terminal deoxynucleotidyl transferase-mediated dUTP nick-end labeling.

Removal from Transplant Waiting List and Recurrence of Heart Failure

Patient #1 was removed from the list of the Japan Organ Transplant Network, because the improvement in cardiac function was great enough for removal at the end of treatment (EF 18 to 29%, BNP 53 to 16 pg/mL). LVAD was removed from Patient #3 after treatment, because the improvement in cardiac function was great enough for removal (EF 8 to 24%, BNP 188 to 50 pg/mL). At the follow-up, he was removed from the list of the Japan Organ Transplant Network, because he was stable even after the discontinuation of mechanical and inotropic support (EF 24 to 38%, BNP 50 to 99 pg/mL). All patients except Patient #6 were discharged from the hospital after the treatment.

The patient #8 was discharged with stable condition eight days after cessation of IGF-1. Seventy-two days after cessation of IGF-1, the patient re-admitted hospital for recurrence of heart failure. Eighty-seven days after cessation of IGF-1, the patients got cardiogenic shock even with intravenous catecholamine. Intra-aortic balloon pumping was not effective for hemodynamic support. Next day, the patient received LVAD implantation. His three-month follow-up study was not conducted because of unstable hemodynamic conditions.

### Discussion

Main findings of the study were 1) three-month administration of recombinant IGF-1 was safe and feasible; 2) symptom of heart failure, daily life activity, ventricular contractility, ventricular mass, exercise and vasodilatory capacity were improved; 3) A part of improvements remained even at the three-month follow-up; 4) Two out of eight patients were removed from the waiting list because of improvement in cardiac function.

Left ventricular Hypertrophy and Functional Improvement

In the present study, exogenous IGF-1 tripled the basal IGF-1 level at the end of treatment. Simultaneously, endogenous GH and IGFBP-3 levels were suppressed. In the previous GH studies, serum level of IGF-1 induced by exogenous GH correlated with LV mass (Isgaard J et al Eur Heart J. 1998; 19:1704-11) and LVEF (Osterziel K J et al Clin Endocrinol (Oxf). 2000; 53:61-8). The present study showed the correlation between the changes in LVEF and LVMI during the IGF-1 treatment, suggesting that induction of cardiac hypertrophy partly underlies the inotropic effects of IGF-1. Echocardiographic diastolic indices and myocardial fibrosis did not deteriorate during the study. Moreover, the changes in myocardial diameter at the end of treatment correlated with the changes in LVMI, suggesting LV hypertrophy induced by IGF-1 was primarily of myocardial, and not fibrotic origin. Thus, the LV hypertrophy observed here upon IGF-1 treatment might constitute physiological rather than pathological hypertrophy, in corroboration with previous studies (Welch S et al Circ Res. 2002; 90:641-8, Rommel C et al Nat Cell Biol. 2001; 3:1009-13).

Myocardial staining area and gene expression of IGF-1 were up-regulated at the end of treatment in spite of tremendous elevation of circulating IGF-1. Although exogenous IGF-1 down-regulates IGF-1 receptors in physiological situation (Bostedt K T et al Exp Cell Res. 2001; 271:368-77), the inventors found the up-regulation of myocardial staining area and gene expression of IGF-1 receptor at the end of treatment. Underlying molecular mechanisms need to be elucidated. Nevertheless, accompaniment of the myocardial staining of phosphorylated Akt kinase, which is in the downstream of IGF-1 receptor, might suggest the IGF-1 signal transduction was initiated by treatment in the diseased myocardium. Further, Akt kinase plays a role in physiological myocardial hypertrophy (Welch S et al Circ Res. 2002; 90:641-8, Rommel C et al Nat Cell Biol. 2001; 3:1009-13). Recent studies have suggested that Akt kinase played a pivotal role in the suppression of myocardial apoptosis induced by ischemia/reperfusion, oxidative stresses, and heart failure (Cittadini A et al. Circ Res. 1998; 83:50-9, Boger R H. J Endocrinol Invest. 1999; 22:75-81, Fujio Y et al Circulation. 2000; 101: 660-7).

The inventors do not know the mechanism of loss of improvements that was obtained by IGF-1 at the follow-up. This may relate to myocyte degradation in dilated cardiomyopathy. Apoptosis, ubiquitin-proteasome proteolysis, lysosomal cathepsin hydrolysis, calcium-dependent calpain digestion and autophagy may be involved in myocardial degradation in dilated cardiomyopathy. IGF-1 hypertrophies cardiac and skeletal myocytes and interrupts the myocyte proteolysis (Komamura K et al Cardiovasc Drugs Ther. 2003; 17:303-10). The balance between myocardial hypertrophy and atrophy determines the left ventricular mass and hence ejection fraction. In dilated cardiomyopathy, myocardial degradation processes may not be completely interrupted with exogenous IGF-1.

### Pro-Inflammatory Cytokines and Apoptosis

Activation of inflammatory cytokines, such as TNF-A and IL-6, has been associated with more severe symptoms and shortened survival in heart failure (Rauchhaus M et al Circulation. 2000; 102:3060-7, Mann D L. Circ Res. 2002; 91:988-98). TNF-ahas been implicated as one of the major modulators of heart failure and has been an important therapeutic target (Kubota T et al Circ Res. 1997; 81:627-35, Torre-Amione G et al Circulation. 1999; 100:1189-93). Previous experiments have demonstrated that TNF-α inhibits the transcriptional response to growth hormone (Anwar A et al Circulation. 2002; 105:1220-5) and suppresses the expression of IGF-1 and its receptor (Fernandez-Celemin L et al Am J Physiol Endocrinol Metab. 2002; 283:E1279-90). On the other hand, IGF-1 is known to be an anti-inflammatory agent that down-regulates pro-inflammatory cytokines (Spies M et al Gene Ther. 2001; 8:1409-15, Adamopoulos S et al Eur Heart J. 2003 December; 24(24):2186-96). A very recent study reported that IGF-1 may interrupt the signaling between TNF-α and pro-apoptotic factors (Dalla Libera L et al Am J Physiol Cell Physiol. 2004; 286:C138-44). In the present study, serum IL-6 and TNF-A levels along with the myocardial staining and gene expression levels of TNF-α decreased at the end of treatment. Those anti-inflammatory effects of exogenous IGF-1 might be relevant to up-regulations of intrinsic IGF-1/IGF-1R system in the diseased myocardium.

### Exercise Tolerance and Peripheral Vessels

Endothelial dysfunction including impairment of endothelial nitric oxide (NO) synthesis is seen in heart failure and may contribute to the exercise intolerance and end-organ dysfunction of chronic heart failure (Ramsey M W et al Circulation 1995; 92:3212-9, Landmesser U et al. Circulation. 2002; 106: 3073-8.). IGF-1 improves endothelial function via an enhancement of endothelial NO synthase activity (Donath MY et al J Clin Endocrinol Metab. 1996; 81:4089-94, Osterziel K J et al Cardiovasc Res. 2000 Jan. 14; 45(2):447-53). These effects of IGF-1 on endothelial function may in part underlie its therapeutic potential in chronic heart failure patients. In the present study, the increases in serum cyclic GMP and nitrite/nitrate levels, and the decreases in serum IL-6 and TNF-A levels accompanied with the improvement in FMD. The improvement in FMD might affect the improvement in peak oxygen consumption. Lastly, the increase in the lean body mass might indicate improvement of skeletal muscle force, e.g. average grip force, which may give rise to the observed improvement in exercise capacity (Table 2).

BNP at the follow-up was still lower than the baseline value. FMD was still improved at the follow-up. Pro-inflammatory cytokines such as TNF-α and IL-6 tended to be lower than baseline values (p=0.063). Sustained improvement of those humoral and vascular factors might keep NYHA functional class or subjective symptom less severe than that in the baseline, even when peak VO2 returned to baseline at the follow-up.

### LIMITATIONS AND CONCLUSIONS

The inventors acknowledge limitations of their study, the foremost of which are the small sample size and lack of randomization and placebo controls. It is ethically hard to administer placebos to end stage patients on the waiting list. This small study needs to be interpreted cautiously. However, the results from the present study are consistent.

In the present study, BNP levels may look rather low for advanced heart failure. Shionogi assay that the inventors used for BNP measurement gives number that can be considerably lower than those from. Triage BNP assay (Biosite Diagnositics, USA) (Fischer Y et al. Clin Chem. 2001; 47:591-4).

One patient, whose cardiac performance responded to the treatment, had an unanticipated severe relapse and needed LVAD. Nonetheless, the present inventors feel that this study demonstrates that short-term recombinant IGF-1 may improve cardiac function and daily life activity of the patients with end-stage DCM. Reports on the relations between IGF-1 and longevity (Holzenberger M et al in mice. Nature. 2003; 421:182-7), cancer (Renehan A G et al Lancet. 2004; 363:1346-53) and atherosclerosis (Delafontaine P et al. Arterioscler Thromb Vasc Biol. 2004; 24:435-44) are accumulating. Long-term and excessive IGF-1 may induce hypertrophic cardiomyopathy and heart failure (Delaughter M C et al FASEB J. 1999; 13:1923-9). Thus, therapeutic usage of IGF-1 should be conducted carefully for a selective subgroup of cardiomyopathy with appropriate dose and duration.

Collectively, our data suggest that three-month IGF-1 therapy is safe and feasible in the patients on the waiting list for heart transplantation with end-stage DCM, and furthermore, is associated with improved cardiac function and symptoms. Thus, IGF-1 therapy might be worth pursuing as a novel method for bridge to transplant or recovery for the patients with end-stage heart failure with idiopathic DCM.

1. A method for improving cardiovascular function and symptoms in a patient with chronic severe heart failure, wherein the method comprises administering therapeutically effective amount of IGF-1 to the patient for a certain period of time, and the improvement of cardiovascular function and symptoms in the patient persists after the cessation of IGF-1 treatment.

2. The method of claim 1, wherein the number of circulating CD34⁺ endothelial progenitor cells in the patient is increased.

3. The method of claim 1, wherein the chronic severe heart failure is accompanied by insulin resistance condition.

4. The method of claim 1, wherein the patient with chronic severe heart failure requires heart transplantation.

5. The method of claim 1, wherein the patient is classified NYHA functional class III through IV.

6. The method of claim 1, wherein the chronic severe heart failure is dilated cardiomyopathy.

7. The method of claim 1, wherein the period is at least three months.

8. The method of claim 1, wherein IGF-1 is administered to the patient once a day.

9. The method of claim 1, wherein the therapeutically effective amount is 0.05 mg/kg to 0.3 mg/kg.

10. The method of claim 1, wherein the amount of IGF-1 administered to the patient is titrated over the period of time for administration.

11. The method of claim 1, wherein the administration is subcutaneous.

12. The method of claim 1, wherein the method further comprises, in the certain period of treatment:
a) monitoring the level of blood sugar in the patient, and;
b) administering sugars to the patient.

13. A pharmaceutical composition for improving cardiovascular function and symptoms in a patient with chronic severe heart failure, comprising therapeutically amount of IGF-1.

14. Use of IGF-1 for manufacturing a medicament for improving cardiovascular function and symptoms in a patient with chronic severe heart failure.
